(19) United States
(12) Patent Application Publication
(10) Pub. No.: US 2018/0042882 A1
(43) Pub. Date: Feb. 15, 2018

(54) C16:1n7-PALMITOLEATE AND DERIVATIVES THEREOF FOR TREATING OBESITY, PROMOTING WEIGHT LOSS, AND SUPPORTING WEIGHT MANAGEMENT

(71) Applicant: Tersus Life Sciences, LLC, Bonita Springs, FL (US)

(72) Inventor: Lochlainn O'HAIMHIRGIN, Mentor, OH (US)

(21) Appl. No.: 15/557,065

(22) PCT Filed: Mar. 8, 2016
(86) PCT No.: PCT/US2016/021372

§ 371 Date: Sep. 8, 2017

Related U.S. Application Data
(60) Provisional application No. 62/130,445, filed on Mar. 9, 2015.

(51) Int. Cl. A61K 31/231

(52) U.S. Cl. CPC A61K 31/231

(57) ABSTRACT

The present technology provides methods for treating obesity, promoting weight loss, and supporting weight management in a subject in need thereof. The methods include administering to the subject an effective amount of a composition comprising one or more of C16:1n7-palmitoleate, derivatives thereof, or pharmaceutically acceptable salts thereof.

### CROSS-REFERENCE TO RELATED APPLICATIONS

This application claims the benefit of and priority to U.S. Application No. 62/130,445, filed Mar. 9, 2015, the contents of which are incorporated herein by reference in their entireties.

### TECHNICAL FIELD

The present technology relates generally to methods for treating obesity, promoting weight loss, and supporting weight management in a subject in need thereof comprising administering to the subject an effective amount of a composition containing C16:1n7-palmitoleate, or derivatives or pharmaceutically acceptable salts thereof.

### BACKGROUND

Excess fat deposition or obesity is a major problem that negatively impacts health, reproduction, and economic resources. More than 17% of children and 34% of adults in the U.S. are considered obese. Obesity is a risk factor for many other co-morbidities including diabetes, hypertension, high cholesterol, stroke, heart disease, breast cancer, and arthritis. The economic costs of human obesity are an estimated $151 billion, an amount which is projected to reach $900 billion by 2030 in the U.S. alone. The 2000 National Beef Quality Audit has estimated that roughly $1.3 billion dollars or 43% of the total quality losses in the beef industry are attributable to excess external fat on beef carcasses.

Studies show that the majority (70%) of fat accumulation in meat producing animals during postnatal growth is due to an increase in hypertrophy or enlarging of adipocytes with excess lipids (Robelin, 1986). Fat accumulation in ruminant adipose tissues is the result of combined adipocyte hyperplasia and hypertrophy. Lipid content in bovine subcutaneous adipose tissues increased from 0.7 to 138 kg and adipocyte diameter increased from 45 to 135 μm from birth to maturity (700 kg BW). Hyperplasia is completed around 8 months of age in bovine subcutaneous adipose tissues; thus the majority of fat accumulation that occurs after 8 months of age is due to hypertrophy or lipid filling (Hood and Allen, 1973).

Accordingly, there is a substantial need for effective methods for treating obesity, promoting weight loss, and supporting weight management.

### SUMMARY OF THE PRESENT TECHNOLOGY

In one aspect, the present technology provides a method for treating obesity, promoting weight loss, and supporting weight management in a subject in need thereof comprising administering to the subject an effective amount of a composition comprising one or more of C16:1n7-palmitoleate, a C16:1n7-palmitoleate derivative, or a pharmaceutically acceptable salt thereof.

In some embodiments, the composition utilized by the methods described herein, such as a nutraceutical or a dietary supplement, comprises between 1% to 100% of C16:1n7-palmitoleate and its derivatives relative to all of the components of the nutraceutical composition. In some embodiments, the composition comprises from about 1% to about 5%, from about 5% to about 10%, from about 10% to about 15%, from about 15% to about 20%, from about 20% to about 25%, from about 25% to about 30%, from about 30% to about 35%, from about 35% to about 40%, or at least about 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of C16:1n7-palmitoleate or one or more derivatives thereof relative to all of the components of the composition.

In some embodiments of the method, the composition also includes C16:0-palmitate, a C16:0-palmitate derivative, or a combination thereof.

Additionally or alternatively, in some embodiments of the method, the composition also includes C18:1n9-oleate, a C18:1n9-oleate derivative, or a combination thereof.

Additionally or alternatively, in some embodiments, the method includes simultaneously, sequentially, or separately administering at least one additional therapeutic agent.

Additionally or alternatively, in some embodiments of the method, the composition is administered orally, topically, systemically, intravenously, subcutaneously, intraperitoneally, or intramuscularly.

In some embodiments of the method, the composition is administered daily for 1, 2, 3, 4 weeks or more.

In some embodiments of the method, the subject exhibits one or more signs or symptoms of obesity including, but not limited to: a body mass index (BMI) over 30 (obese subject), a BMI between 25-30 (overweight subject), a daily average weight pain, increase in adipocyte cell size, increased rate of fat deposition, increased rate of lipidogenesis, and reduced rate of lipid oxidation. In some embodiments, the subject is human.

In some embodiments, administration of the composition causes a reduction in BMI in the subject. In some embodiments, the subject's BMI is reduced to 18.5-25 following administration of the composition.

In some embodiments, administration of the composition causes a reduction in the mean cell size of adipocytes in the subject. In some embodiments of the method, the mean cell size of adipocytes in the subject is reduced within one month following the administration of the composition to the subject. In some embodiments of the method, the mean cell size of adipocytes in the subject is reduced from about 1% to about 30% within one month following the administration of the composition to the subject. In some embodiments of the method, the mean cell size of adipocytes in the subject is reduced by about 10% within one month following the administration of the composition to the subject.

In some embodiments, administration of the composition causes a reduction in the rate of fat deposition in the subject. in some embodiments of the method, the rate of fat deposition in the subject is reduced within one month following the administration of the composition to the subject. In some embodiments of the method, the rate of fat deposition in the subject is reduced from about 1% to about 50% within one month following the administration of the composition to the subject.

In some embodiments, administration of the composition causes a reduction in the rate of lipidogenesis in the subject. In some embodiments of the method, the rate of lipidogenesis in the subject is reduced within one month following the administration of the composition to the subject. In some embodiments of the method, the rate of lipidogenesis in the subject is reduced from about 1% to about 50% within one month following the administration of the composition to the subject.

In some embodiments, administration of the composition causes an increase in the rate of lipid oxidation in the subject. In some embodiments of the method, the rate of lipid oxidation in the subject is increased within one month following the administration of the composition to the subject. In some embodiments of the method, the rate of lipid oxidation in the subject is increased from about 1% to about 50% within one month following the administration of the composition to the subject.

In some embodiments of the method, the appetite of the subject is not substantially affected following the administration of the composition to the subject.

### BRIEF DESCRIPTION OF THE DRAWINGS

FIG. 1 illustrates de novo lipogenesis and β-oxidation in an adipocyte.

FIG. 2 illustrates de novo lipogenesis and β-oxidation in an adipocyte after exogenous palmitoleic acid addition.

### DETAILED DESCRIPTION

It is to be appreciated that certain aspects, modes, embodiments, variations and features of the present technology are described below in various levels of detail in order to provide a substantial understanding of the present technology.

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a”, “an” and “the” include plural referents unless the content clearly dictates otherwise. For example, reference to “a compound” includes a plurality of compounds.

As used herein, the terms “approximately” or “about” in reference to a number are generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

As used herein, the term “composition” includes therapeutic and dietary formulations including, but not limited to a dietary supplement, nutraceutical formulation, or pharmaceutical formulation. Compositions comprising C16:1n7-palmitoleate include dietary supplements, nutraceutical formulations, and pharmaceutical compositions. “Pharmaceutically acceptable composition” refers to a composition that is suitable for administration to a subject, particularly, a human. Such compositions include various excipients, diluents, carriers, and such other inactive agents well known to the skilled artisan. In another aspect, any of the pharmaceutical compositions, as described in the published U.S. Patent Application US 2012/0225941, incorporated herein by reference in its entirety, are provided where the pharmaceutical compositions include C16:In7-palmitoleate or any derivatives thereof.

The methods described herein utilize compositions that include C16:1n7-palmitoleate or any one or more derivatives thereof as described in the U.S. Pat. No. 8,703,818, which is incorporated herein by reference in its entirety. In some embodiments, the C16:1n7-palmitoleate derivative is C16:1n7-palmitoleic acid. In further embodiments, the C16:1n7-palmitoleate derivative is cis-C16:1n7-palmitoleic acid. In some embodiments, the C16:1n7-palmitoleate derivative is a metal salt (e.g., Na⁺, K⁺, or Li⁺) of cis-C16:1n7-palmitoleate. In further embodiments, the C16:1n7-palmitoleate derivative is an ester (e.g., (C₁-C₈) alkyl ester, methyl, ethyl, propyl, monoglyceride, diglyceride, triglyceride, or a combination thereof.) of cis-C16:1n7-palmitoleate. In further embodiments, the C16:1n7-palmitoleate derivative is a methyl ester, ethyl ester, propyl ester of cis-C16:1n7-palmitoleate. In one embodiment, the cis-C16:1n7-palmitoleate ester is the ethyl ester.

The methods described herein are not limited to any particular chemical form of C16:1n7-palmitoleate and the compound may be given to subjects either as an ester, free acid or as a pharmaceutically acceptable salt.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease or medical condition, a positive control (a compound or composition known to exhibit the desired therapeutic effect) or a negative control (a subject or a sample that does not receive the therapy or receives a placebo) is typically employed.

As used herein, the term “effective amount” refers to a quantity sufficient to achieve a desired therapeutic and/or prophylactic effect, e.g., an amount which results in the prevention or treatment of obesity, the promotion of weight loss, and the support of weight management. In the context of therapeutic or prophylactic applications, the amount of a composition administered to the subject will depend on the degree, type and severity of the disease or medical condition and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, C16:1n7-palmitoleate (or derivatives, pharmaceutically acceptable salts, or a combination thereof) may be administered to a subject having one or more signs or symptoms of obesity. For example, a “therapeutically effective amount” of C16:1n7-palmitoleate (or derivatives, pharmaceutically acceptable salts, or a combination thereof) means levels at which the physiological effects of obesity are, at a minimum. ameliorated. A therapeutically effective amount can be given in one or more administrations. In some embodiments, signs, symptoms or complications of obesity include, but are not limited to: daily average weight gain, increase in adipocyte cell size, increased rate of fat deposition, increased rate of lipidogenesis, reduced rate of lipid oxidation, breathing disorders, coronary artery disease, diabetes, high blood pressure, high cholesterol and stroke. In one embodiment, the therapeutically effective amount of the compound is from about 500 to about 3000 mg per day. In another embodiment, the therapeutically effective amount of the compound is from about 1000 to about 2000 mg per day. In one embodiment, the therapeutically effective amount of the compound is from about 0.01 to about 1000 mg per day. In another embodiment, the therapeutically effective amount of the compound is from about 0.1 to about 500 mg per day. In another embodiment, the therapeutically effective amount of the compound is from about 1.0 to about 200 mg per day. In yet another embodiment, the therapeutically effective amount of the compound is from about 10 to about 100 mg per day.

As used herein, the term “monoglyceride” refers to a fatty acid chain, such as C16:1n7-palmitoleate, covalently bonded to a glycerol molecule through an ester linkage. As used herein, the term “diglyceride” refers to a fatty acid chain, such as C16:1n7-palmitoleate, covalently bonded to a glycerol molecule through an ester linkage, wherein the glycerol molecule is further bonded to one additional fatty acid chain, which may or may not be C16:1n7-palmitoleate, though one additional ester linkage. As used herein, the term “triglyceride” refers to a fatty acid chain, such as C16:1n7-palmitoleate, covalently bonded to a glycerol molecule through an ester linkage, wherein the glycerol molecule is further bonded to two additional fatty acid chains, either or both of which may or may not be C16:1n7-palmitoleate, though two additional ester linkages.

As used herein, the term “obesity” refers to a chronic condition defined by an excess amount of body fat. Obesity is best defined by using the body mass index. The body mass index (BMI) equals a person's weight in kilograms (kg) divided by their height in meters (m) squared. Since BMI describes body weight relative to height, it strongly correlates with total body fat content in adults. An adult who has a BMI of 25-29.9 is considered overweight, whereas an adult who has a BMI over 30 is considered obese.

As used herein, “prevention” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the terms “subject,” “individual,” or “patient” can be an individual organism, a vertebrate, a mammal, or a human.

A “therapeutic level” of a drug is an amount of C16:1n7-palmitoleate or a C16:1n7-palmitoleate derivative that is sufficient to treat or prevent obesity but not high enough to pose any significant clinical risk to the patient. Therapeutic levels of drugs can he determined by tests that measure the actual concentration of the compound in the blood of the patient. This concentration is referred to as the “serum concentration.”

As used herein, the term “therapeutic use” of the compounds discussed herein is defined as using one or more of the compounds discussed herein to treat or prevent obesity, promote weight loss, and support weight management.

“Treating” or “treatment” as used herein covers the treatment of a disease or medical condition described herein (i.e., obesity), in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or medical condition.

As used herein, the term “weight loss” is defined as a reduction of the total body weight. Weight loss may for example refer to the loss of total body mass in an effort to improve fitness, health, and/or appearance.

As used herein, the terms “weight management” or “weight maintenance” relates to maintaining a total body weight. For example, weight management or weight maintenance may relate to maintaining a BMI in the area of 18.5-25, which is considered to be

It is also to be appreciated that the various modes of treatment or prevention of obesity as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

### General

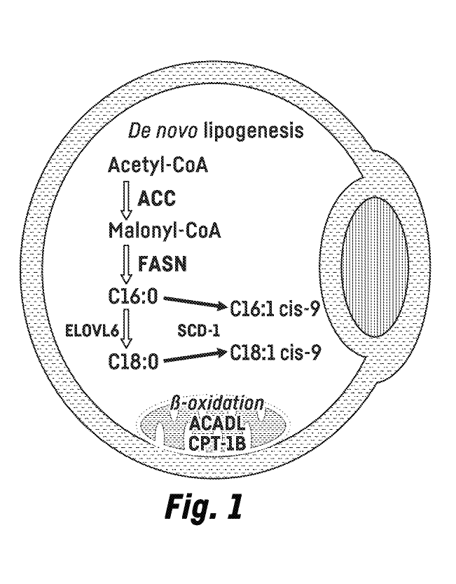

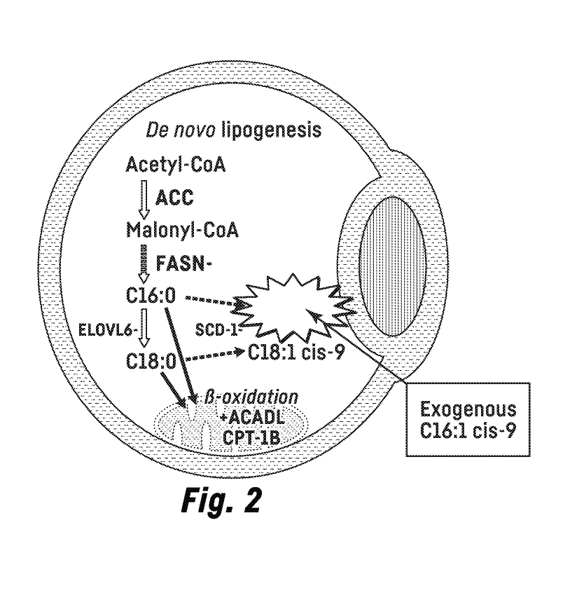

Fatty acids available for lipid filling come from two sources, either dietary sources or de nova lipogenesis. In ruminants, adipose tissue is the primary site for lipid filling (over 90% in adipose tissues) and acetate is the predominant carbon source for de novo lipogenesis (Ingle et al., 1972a,b). However in rodents, the liver serves as the primary site of lipogenesis and glucose is the primary carbon source (Ntambi et al. 1992). The key enzymes involved in de novo fatty acid synthesis are: acetyl-CoA carboxylase and fatty acid synthase. As shown in FIGS. 1 and 2, acetyl-CoA carboxylase (ACC) catalyzes the first committed step in de novo fatty acid synthesis. Fatty acid synthase (FASN) is an enzyme complex that adds carbons to malonyl-CoA to form palmitoleic acid (C16:0). Palmitic acid can be further elongated to stearic acid (C18:0) by fatty acid elongase (ELOVL-6). Palmitic and stearic acids can be desaturated to palmitoleic acid (C16:1) and oleic acid (C18:1) by stearoyl-CoA desaturase (SCD-1). Palmitoleic and oleic acids are the principal end products of SCD-1 and represent the majority (98%) of monounsaturated fatty acids (MUFAs) in adipose tissues of meat producing animals. Stearoyl-CoA desaturase, also known as the Δ9 desaturase, is the rate-limiting enzyme in de novo synthesis of MUFAs. SCD-1 expression directly regulates the ratio of saturated fatty acids to MUFAs and membrane fluidity, thereby maintaining normal cell function.

### Compositions

In some embodiments, the composition includes C16:1n7-palmitoleate, or derivatives, pharmaceutically acceptable salts, or a combination thereof. Compositions that include C16:1n7-palmitoleate and its derivatives to be utilized in the methods described herein include any of those described in U.S. Pat. No. 8,703,818 which is incorporated herein by reference in its entirety.

In some embodiments, the composition utilized by the methods described herein, such as a nutraceutical, pharmaceutical, or a dietary supplement, comprises between 1% to 100% of C16:1n7-palmitoleate and its derivatives relative to all of the components of the nutraceutical composition. In some embodiments, the composition comprises from about 1% to about 5%, from about 5% to about 10%, from about 10% to about 15%, from about 15% to about 20%, from about 20% to about 25%, from about 25% to about 30%, from about 30% to about 35%, from about 35% to about 40%, or at least about 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of C16:1n7-palmitoleate or one or more derivatives thereof relative to all of the components of the composition.

In some embodiments, the composition comprises a C16:1n7-palmitoleate derivative, wherein the wt % of the C16:1n7-palmitoleate derivative exceeds the wt % of any other single ingredient in the composition. In some embodiments, the composition comprises at least about 50 wt % of the C16:1n7-palmitoleate derivative. In some embodiments, the composition comprises at least about 60 wt % of the C16:1n7-palmitoleate derivative. In some embodiments, the composition comprises at least about 70 wt % of the C16:1n7-palmitoleate derivative. In some embodiments, the composition comprises at least about 80 wt % of the C16:1n7-palmitoleate derivative. In some embodiments, the composition comprises at least about 90 wt % of the C16:1n7-palmitoleate derivative.

Additionally or alternatively, in some embodiments, the composition, such as a nutraceutical, pharmaceutical, or a dietary supplement, comprises about 1% to about 100% of C16:1n7-palmitoleate and its derivatives relative to all of the fatty acids and fatty acid derivatives that are present in the composition. In some embodiments, the composition comprises from about 5% to about 20%, from about 20% to about 30%, or at least about 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of C16:1n7-palmitoleate or one or more derivatives thereof relative to all of the fatty acids and fatty acid derivatives that are present in the composition.

In certain embodiments, the composition, such as a nutraceutical, pharmaceutical, or a dietary supplement, comprises C16:1n7-palmitoleate and its derivatives and further comprises C16:1n7-palmitate and its derivatives. In certain embodiments, the composition comprises C16:1n7-palmitoleate and its derivatives relative to C16:0-palmitate and its derivatives in a ratio in excess of 1:1. In certain embodiments, the composition comprises a ratio of C16:1n7-palmitoleate and its derivatives relative to C16:0-palmitate and its derivatives (i.e., palmitoleate:palmitate), wherein the ratio is about 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1,5.4:1, 5.5:1, 5,6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8,8:1, 8.9:1, 90:1, 9.1:1, 91:1, 9.3:1, 9.4:1, 9.5:1, 9.6:1, 9.7:1, 9.8:1, 9.9:1, 10.0:1, 11:1, 12:1, 13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, 100:1, 200:1 or a ratio between any two of those recited above.

In some embodiments, the composition comprises a C16:1n7-palmitoleate derivative and a palmitate derivative, wherein the ratio of the C16:1n7-palmitoleate derivative to the palmitate derivative (i.e., palmitoleate:palmitate) is from about 12:1 to about 100:1; and each palmitoleate and palmitate derivative is independently selected from the group consisting of a free acid, pharmaceutically acceptable salt, (C₁-C₈) alkyl ester, monoglyceride, diglyceride, triglyceride and a combination thereof In some embodiments, the ratio of the C16:1n7-palmitoleate derivative to the palmitate derivative is from about 15:1 to about 50:1. In some embodiments, the ratio of the C16:1n7-palmitoleate derivative to the palmitate derivative is from about 50:1 to about 100:1.

In some embodiments, all of the palmitoleate and palmitate derivatives are (C₁-C₈) alkyl esters. In some embodiments, all of the palmitoleate and palmitate derivatives are ethyl esters. In some embodiments, all of the palmitoleate and palmitate derivatives are methyl esters. In some embodiments, all of the palmitoleate and palmitate derivatives are propyl, butyl, pentyl, hexyl, heptyl or octyl esters. In some embodiments, all of the palmitoleate and palmitate derivatives are free acids or pharmaceutically acceptable salts thereof In some embodiments, all of the palmitoleate and palmitate derivatives are selected from the group consisting of monoglycerides, diglycerides, triglycerides and combinations thereof. In some embodiments, the C16:1n7-palmitoleate derivative is a cis-C16:1n7-palmitoleate derivative.

In certain embodiments, the composition, such as a nutraceutical, pharmaceutical, or a dietary supplement, comprises C16:1n7-palmitoleate and its derivatives and further comprises C18:1n9-oleate or its derivatives. In certain embodiments, the composition comprises C16:1n7-palmitoleate and its derivatives relative to C18:1n9-oleate and its derivatives in a ratio in excess of 1:1.

In some embodiments, the composition, such as a nutraceutical, pharmaceutical, or a dietary supplement, comprises C16:1n7-palmitoleate and its derivatives and further comprises C1.8:1n9-oleate and its derivatives, wherein the ratio of the C16:1n7-palmitoleate derivative to the oleate derivative (i.e., palmitoleate: oleate) is from about 1.1:1 to about 100:1. In some embodiments, the composition comprises a ratio of C16:1n7-palmitoleate and its derivatives relative to C18:1n9-oleate and its derivatives (i.e., palmitoleate:oleate), wherein the ratio is about 1.1:1, 1.2:1, 1.3:1, 1.4:1, 1.5:1, 1.6:1, 1.7:1, 1.8:1, 19:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1,4.1:1, 4.2:1,4.3:1, 4.4:1, 4.5:1, 4.6:1, 4.7:1, 4,8:1, 4.9:1, 5.0:1, 5.1:1, 5.2:1, 5,3:1, 5.4:1, 5,5:1, 5.6:1, 5,7:1, 5.8:1, 5.9:1, 6.0:1, 6.1:1, 6,2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7.6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 94:1, 9.5:1, 96:1, 9.7:1, 9.8:1, 9.9:1, 10.0:1, 11:1, 13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, 100:1, 200:1 or a ratio between any two of those recited above.

In certain embodiments, the composition further comprises an oleate derivative, wherein the ratio of the C16:1n7-palmitoleate derivative to the oleate derivative is from about 6:1 to about 100:1, and each oleate derivative is independently selected from the group consisting of a free acid, pharmaceutically acceptable salt, (C₁-C₈) alkyl ester, monoglyceride, diglyceride, triglyceride and a combination thereof In certain embodiments, the ratio of the C16:1n7-palmitoleate derivative to the oleate derivative is from about 10:1 to about 20:1. In certain embodiments, the ratio of the C16:1.n7-palmitoleate derivative to the oleate derivative is from about 20:1 to about 50:1. In certain embodiments, the ratio of the C16:1n7-palmitoleate derivative to the oleate derivative is from about 50:1 to about 100:1.

Additionally or alternatively, in any of the above embodiments, the composition, such as a nutraceutical, pharmaceutical, or a dietary supplement, comprises C16:1n7-palmitoleate and its derivatives and further comprises C18:1n7-vaccenoate or its derivatives. In certain embodiments, the composition comprises C16:1n7-palmitoleate and its derivatives relative to C18:1n7-vaccenoate and its derivatives in a ratio in excess of 1:1. In some embodiments, the composition comprises a ratio of C16:1n7-palmitoleate and its derivatives relative to C18:1n7-vaccenoate and its derivatives (i.e., palmitoleate:C18:1n7-vaccenoate), wherein the ratio is in excess of 1.1:1, 12:1, 1.3:1, 1.4:1, 1.5:1, 1.6:1, 1,7:1, 1.8:1, 1.9:1, 2.0:1, 2.1:1, 2.2:1, 2.3:1, 2,4:1, 2.5:1, 2,6:1, 2.7:1, 2,8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, 7.0:1, 7.1:1, 7.2:1, 7.3:1, 7.4:1, 7.5:1, 7,6:1, 7.7:1, 7.8:1, 7.9:1, 8.0:1, 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1, 9.5:1, 9.6:1, 9.7:1, 9.8:1, 9.9:1, 10.0:1, 11:1, 12:1, 13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, or 100:1.

In certain embodiments, the composition further comprises a C18:1n7-vaccenoate derivative, wherein the ratio of the C16:1n7-palmitoleate derivative to the C18:1n7-vaccenoate derivative is from about 3:1 to about 100:1, and each C18:1n7-vaccenoate derivative is independently selected from the group consisting of a free acid, pharmaceutically acceptable salt, (C₁-C₈) alkyl ester, monoglyceride, diglyceride, triglyceride and a combination thereof. In certain embodiments, the ratio of the C16:1n7-palmitoleate derivative to the C18:1n7-vaccenoate derivative is from about 5:1 to about 20:1. In certain embodiments, the ratio of the C16:1n7-palmitoleate derivative to the C18:1n7-vaccenoate derivative is from about 20:1 to about 50:1. In certain embodiments, the ratio of the C16:1n7-palmitoleate derivative to the C18:1n7-vaccenoate derivative is from about 50:1 to about 100:1.

In another embodiment, a composition contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight, palmitic acid, if any. In another embodiment, a composition contains substantially no palmitic acid. In still another embodiment, a composition contains no palmitic acid and/or derivative thereof.

C16:1n7-palmitoleate and derivatives thereof (also known as omega-7), for use in the methods described herein, can be obtained from any of the sources and methods described in U.S. Pat. No. 8,703,818, which is incorporated herein by reference in its entirety. In certain embodiments, C16:1n7-palmitoleate and derivatives thereof are isolated, concentrated, and/or purified from a source selected from the group consisting of one or more plants, animals, fish, and microorganisms. In other embodiments, the C16:1n7-palmitoleate moiety of the C16:1n7-palmitoleate derivative is obtained from a source selected from the group consisting of fish, macadamia nuts, sea buckthorn, tallow, algae, bacteria, yeast, and a combination thereof.

In some embodiments, the C16:1n7-palmitoleate derivative comprises a C16:1n7-palmitoleate moiety that is obtained from fish. In some embodiments, the fish is selected from the group consisting of anchovies, menhaden, pollock, herring, cod, salmon, smelt, tuna, mackerel, krill and a combination thereof. In some embodiments, the fish comprise anchovies. In other embodiments, the fish comprise menhaden.

### Methods for Preventing or Treating Obesity, Promoting Weight Loss, and Supporting Weight Management

The present technology provides methods for preventing or treating obesity, promoting weight loss, and supporting weight management in a subject comprising administering to the subject an effective amount of a composition comprising C16:1n7-palmitoleate, derivatives thereof, pharmaceutically acceptable salts thereof, or a combination thereof. In some embodiments, the method includes administering to the subject one or more of any one of the embodiments of the composition described herein.

The compositions comprising C16:1n7-palmitoleate, derivatives thereof, pharmaceutically acceptable salts thereof, or a combination thereof described herein are useful to prevent or treat obesity, promote weight loss, and support weight management.

Compositions comprising C16:1n7-palmitoleate, derivatives thereof, pharmaceutically acceptable salts thereof, or a combination thereof, such as those described above, (e.g., C16:1n7-palmitoleate alone or C16:1 n7-palmitoleate combined with C18:1n9-oleate) are useful in treating obesity, as well as the signs, symptoms or complications of obesity, promoting weight loss, and supporting weight management.

The disclosure also provides for both prophylactic and therapeutic methods of treating a subject having or at risk for (or susceptible to) obesity. Accordingly, the present methods provide for the prevention and/or treatment of obesity, the promotion of weight loss, and the support of weight management in a subject by administering an effective amount of a composition comprising C16:1n7-palmitoleate, derivatives thereof, pharmaceutically acceptable salts thereof, or a combination thereof to a subject in need thereof.

### Therapeutic Methods

One aspect of the present technology includes methods of treating obesity, promoting weight loss, and supporting weight management in a subject in need thereof. One aspect of the present technology includes methods of treating obesity in a subject diagnosed as being, or at risk of becoming obese. In therapeutic applications, compositions or medicaments comprising C16:1n7-palmitoleate, derivatives thereof, pharmaceutically acceptable salts thereof, or a combination thereof are administered to a subject suspected of, or already suffering from obesity in an amount sufficient to cure, or at least partially arrest, the symptoms of the disease or medical condition, including its complications and intermediate pathological phenotypes in development of the disease or medical condition.

Subjects suffering from obesity can be identified by any or a combination of diagnostic or prognostic assays known in the art. For example, typical symptoms of obesity include, but are not limited to, symptoms such as, e.g., daily average weight gain, increase in adipocyte cell size, increased rate of fat deposition, increased rate of lipidogenesis, and reduced rate of lipid oxidation.

In some embodiments, the subject may exhibit daily average weight gain, increase in adipocyte cell size, increased rate of fat deposition, increased rate of lipidogenesis, and reduced rate of lipid oxidation, which are measurable using techniques known in the art.

### Prophylactic Methods

In one aspect, the present technology provides a method for preventing or delaying the onset of obesity or symptoms of obesity, and supporting weight management in a subject in need thereof. Subjects at risk for obesity can be identified by, e.g., any or a combination of diagnostic or prognostic assays known in the art. In prophylactic applications, compositions or medicaments comprising C16:1n7-palmitoleate, derivatives thereof, pharmaceutically acceptable salts thereof, or a combination thereof are administered to a subject susceptible to, or otherwise at risk for obesity in an amount sufficient to eliminate or reduce the risk, or delay the outset of obesity, and support weight management, including biochemical, histologic and/or behavioral symptoms of the disease or disorder, its complications and intermediate pathological phenotypes presenting during development of the disease. Administration of a prophylactic compositions or medicaments comprising C16:1n7-palmitoleate, its derivatives, pharmaceutically acceptable salts thereof, or a combination thereof can occur prior to the manifestation of symptoms characteristic of the disease or disorder, such that the disease or disorder is prevented or, alternatively, delayed in its progression.

Subjects at risk for obesity may exhibit one or more of the following non-limiting risk factors: excessive caloric intake (overeating), low levels of physical activity, stress, anxiety, lack of sleep, pregnancy, menopause, medications such as steroids, birth control pills and antidepressants, and medical conditions such as Cushing syndrome, Prader-Willi syndrome and Polycystic ovarian syndrome.

For therapeutic and/or prophylactic applications, a composition comprising one or more of C16:1n7-palmitoleate, a C16:1n7-palmitoleate derivative, or a pharmaceutically acceptable salt thereof, is administered to the subject. In some embodiments, the composition is administered one, two, three, four, or five times per day. In some embodiments, the composition is administered more than five times per day. Additionally or alternatively, in some embodiments, the composition is administered every day, every other day, every third day, every fourth day, every fifth day, or every sixth day. In some embodiments, the composition is administered weekly, bi-weekly, tri-weekly, or monthly. In some embodiments, the composition is administered for a period of one, two, three, four, or five weeks. In some embodiments, the composition is administered for six weeks or more. In some embodiments, the composition is administered for twelve weeks or more. In some embodiments, the composition is administered for a period of less than one year. In some embodiments, the composition is administered for a period of more than one year.

In some embodiments, the composition is administered daily for one week or more. In some embodiments, the composition is administered daily for 2 weeks or more. In some embodiments, the composition is administered daily for 3 weeks or more. In some embodiments, the composition is administered daily for 4 weeks or more. In some embodiments, the composition is administered daily for 6 weeks or more. in some embodiments, the composition is administered daily for 12 weeks or more. In some embodiments, the composition is administered daily for 1 year or more.

### Use of C16:1n7-Palmitoleate to Prevent, Ameliorate or Treat Obesity, Promote Weight Loss, and Support Weight Management

In one aspect, the present technology provides a method for treating or preventing obesity, promoting weight loss, and supporting weight management in a subject in need thereof comprising administering to the subject an effective amount of a composition comprising one or more of C16:1n7-palmitoleate, a C16:1n7-palmitoleate derivative, or a pharmaceutically acceptable salt thereof in some embodiments of the method, the composition also includes C16:0-palmitate, a C16:0-palmitate derivative, or a combination thereof Additionally or alternatively, in some embodiments of the method, the composition also includes C18:1n9-oleate, a C18:1n9-oleate derivative, or a combination thereof

In some embodiments, the subject exhibits one or more signs or symptoms of obesity including, but not limited to: daily average weight gain, increase in adipocyte cell size, increased rate of fat deposition, increased rate of lipidogenesis, and reduced rate of lipid oxidation.

In some embodiments, administration of the composition causes a reduction in daily average weight gain in the subject. In some embodiments, the daily average weight gain in the subject is reduced by at least 20%. In some embodiments, the daily average weight gain in the subject is reduced by at least 30%. In some embodiments, the daily average weight gain in the subject is reduced by at least 50%. In some embodiments, the daily average weight gain in the subject is reduced by at least 75%. In some embodiments, the daily average weight gain in the subject is reduced by at least 95%.

In some embodiments of the method, the daily average weight gain in the subject is reduced within one month following the administration of the composition to the subject. In some embodiments of the method, the daily average weight gain in the subject is reduced from about 25% to about 100% within one month following the administration of the composition to the subject. In some embodiments of the method, the daily average weight gain in the subject is reduced by about 75% within one month following the administration of the composition to the subject.

In some embodiments, administration of the composition causes a reduction in the mean cell size of adipocytes in the subject. In some embodiments, the mean cell size of adipocytes in the subject is reduced by at least 1%. In some embodiments, the mean cell size of adipocytes in the subject is reduced by at least 10%. In some embodiments, the mean cell size of adipocytes in the subject is reduced by at least 15%. In some embodiments, the mean cell size of adipocytes in the subject is reduced by at least 20%. In some embodiments, the mean cell size of adipocytes in the subject is reduced by at least 25%. In some embodiments, the mean cell size of adipocytes in the subject is reduced by at least 30%. In some embodiments of the method, the adipocytes are intramuscular adipocytes.

In some embodiments of the method, the mean cell size of adipocytes in the subject is reduced within one month following the administration of the composition to the subject. In some embodiments of the method, the mean cell size of adipocytes in the subject is reduced from about 1% to about 30% within one month following the administration of the composition to the subject. In some embodiments of the method, the mean cell size of adipocytes in the subject is reduced by about 10% within one month following the administration of the composition to the subject.

In some embodiments, administration of the composition causes a reduction in the rate of fat deposition in the subject. In some embodiments, the rate of fat deposition in the subject is reduced by at least 10%. In some embodiments, the rate of fat deposition in the subject is reduced by at least 15%. In some embodiments, the rate of fat deposition in the subject is reduced by at least 25%. In some embodiments, the rate of fat deposition in the subject is reduced by at least 35%. In some embodiments, the rate of fat deposition in the subject is reduced by at least 50%.

In some embodiments of the method, the rate of fat deposition in the subject is reduced within one month following the administration of the composition to the subject. In some embodiments of the method, the rate of fat deposition in the subject is reduced from about 1% to about 50% within one month following the administration of the composition to the subject.

In some embodiments, administration of the composition causes a reduction in the rate of lipidogenesis in the subject. In some embodiments, the rate of lipidogenesis in the subject is reduced by at least 10%. In some embodiments, the rate of lipidogenesis in the subject is reduced by at least 15%. In some embodiments, the rate of lipidogenesis in the subject is reduced by at least 25%. In some embodiments, the rate of lipidogenesis in the subject is reduced by at least 35%. In some embodiments, the rate of lipidogenesis in the subject is reduced by at least 50%.

In some embodiments of the method, the rate of lipidogenesis in the subject is reduced within one month following the administration of the composition to the subject. In some embodiments of the method, the rate of lipidogenesis in the subject is reduced from about 1% to about 50% within one month following the administration of the composition to the subject.

In some embodiments, administration of the composition causes an increase in the rate of lipid oxidation in the subject. In some embodiments, the rate of lipid oxidation in the subject is increased by at least 10%. In some embodiments, the rate of lipid oxidation in the subject is increased by at least 15%. In some embodiments, the rate of lipid oxidation in the subject is increased by at least 25%. In some embodiments, the rate of lipid oxidation in the subject is increased by at least 35%. In some embodiments, the rate of lipid oxidation in the subject is increased by at least 50%.

In some embodiments of the method, the rate of lipid oxidation in the subject is increased within one month following the administration of the composition to the subject. In some embodiments of the method, the rate of lipid oxidation in the subject is increased from about 1% to about 50% within one month following the administration of the composition to the subject.

In some embodiments of the method, the appetite of the subject is not substantially affected following the administration of the composition to the subject.

### Modes of Administration and Effective Dosages

Any method known to those in the art for contacting a cell, organ or tissue with the compositions of the present technology (i.e., C16:1n7-palmitoleate or derivatives or pharmaceutically acceptable salts thereof) may be employed. Suitable methods include in vitro, ex vivo, or in vivo methods. In vivo methods typically include the administration of C16:1n7-palmitoleate or derivatives or pharmaceutically acceptable salts thereof, such as those described above, to a mammal, suitably a human. When used in vivo for therapy, C16:1n7-palmitoleate or derivatives or pharmaceutically acceptable salts thereof may be administered to the subject in effective amounts (i.e., amounts that have desired therapeutic effects). The dose and dosage regimen will depend upon the degree of the medical condition in the subject, the characteristics of C16:1n7-palmitoleate or derivatives or pharmaceutically acceptable salts thereof, e.g., its therapeutic index, the subject, and the subject's history.

The effective amount may be determined during pre-clinical trials and clinical trials by methods familiar to physicians and clinicians. An effective amount of C16:1n7-palmitoleate (or derivatives or pharmaceutically acceptable salts thereof) useful in the methods may be administered to a mammal in need thereof by any of a number of well-known methods for administering pharmaceutical compounds. C16:1n7-palmitoleate (or derivatives or pharmaceutically acceptable salts thereof) may be administered systemically or locally.

C16:1n7-palmitoleate (or derivatives thereof) may be formulated as a pharmaceutically acceptable salt. The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primly, secondary and tertiary amines, including substituted amines, cyclic amines, naturally-occurring amines and the like, such as arginine, betaine, caffeine, choline. N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and. trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like.

C16:1n7-palmitoleate or derivatives or pharmaceutically acceptable salts thereof described herein can be incorporated into pharmaceutical compositions for administration, alone or in combination, and administered to a subject for the treatment or prevention of obesity, the promotion of weight loss, and the support of weight management. Such compositions typically include the active agent (e.g., C.16:1n7-palmitoleate) and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must he sterile and should be fluid to the extent that easy syringability exists. It should be stable under the conditions of manufacture and storage and must be preserved against the contaminating action of microorganisms such as bacteria and fungi.

Compositions containing C16:1n7-palmitoleate (or derivatives or pharmaceutically acceptable salts thereof) can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can he brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active compound plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, C16:1n7-palmitoleate (or derivatives or pharmaceutically acceptable salts thereof) can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of C16:1n7-palmitoleate (or derivatives or pharmaceutically acceptable salts thereof) as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

C16:1n7-palmitoleate (or derivatives or pharmaceutically acceptable salts thereof) can be formulated in a carrier system. The carrier can be a colloidal system. The colloidal system can be a liposome, a phospholipid bilayer vehicle. One skilled in the art would appreciate that there are a variety of methods to prepare liposomes. See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. in one embodiment, C16:1n7-palmitoleate (or derivatives or pharmaceutically acceptable salts thereof) can he embedded in the polymer matrix. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.).

in some embodiments, C16:1n7-palmitoleate (or derivatives or pharmaceutically acceptable salts thereof) are prepared with carriers that will protect C16:1n7-palmitoleate (or derivatives or pharmaceutically acceptable salts thereof) against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811. C16:1n7-palmitoleate (or derivatives or pharmaceutically acceptable salts thereof) can also be formulated to enhance intracellular delivery.

Dosage, toxicity and therapeutic efficacy of C16:1n7-palmitoleate (or derivatives or pharmaceutically acceptable salts thereof)can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. in some embodiments, C16:1n7-palmitoleate, or derivatives or pharmaceutically acceptable salts thereof, exhibit high therapeutic indices.

The dosage of such compounds lies within a range of circulating concentrations that include the ED₅₀ with little or no toxicity. The dosage may vary within this range depending upon the dosage form employed and the route of administration utilized. For C16:1n7-palmitoleate (or derivatives or pharmaceutically acceptable salts thereof) used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of C16:1n7-palmitoleate (or derivatives or pharmaceutically acceptable salts thereof), sufficient for achieving a therapeutic or prophylactic effect, ranges from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of C16:1n7-palmitoleate (or derivatives or pharmaceutically acceptable salts thereof) ranges from 0.001-10,000 micrograms per kg body weight. In one embodiment, C16:1n7-palmitoleate (or derivatives or pharmaceutically acceptable salts thereof) concentrations in a carrier mange from 0.2 to 2000 micrograms per delivered milliliter. An exemplary treatment regime entails administration once per day or once a week. In therapeutic applications, a relatively high dosage at relatively short intervals is sometimes required until progression of the medical condition is reduced or terminated, and until the subject shows partial or complete amelioration of symptoms of the medical condition. Thereafter, the patient can be administered a prophylactic regime.

In some embodiments, a therapeutically effective amount of C16:1n7-palmitoleate (or derivatives or pharmaceutically acceptable salts thereof) may be defined as a concentration of C16:1n7-palmitoleate (or derivatives or pharmaceutically acceptable salts thereof) at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would he optimized to maintain the therapeutic concentration at the target tissue. In some embodiments, the doses are administered by single daily or weekly administration, but may also include continuous administration (e.g., parenteral infusion or transdermal application). In some embodiments, the dosage of C16:1n7-palmitoleate (or derivatives or pharmaceutically acceptable salts thereof) is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

In some embodiments, C16:1n7-palmitoleate, or derivatives or pharmaceutically acceptable salts thereof, are administered in an amount that achieves a serum concentration of about 100 ng/ml to about 9000 ng/ml, in another embodiment, C16:1n7-palmitoleate, or derivatives or pharmaceutically acceptable salts thereof, are administered in an amount that achieves a serum concentration of about 100 ng/ml to about 1000 ng/ml. In other embodiments, the serum concentration achieved is about 300 ng/ml to about 500 ng/ml, about 100 ng/ml to about 500 ng/ml, about 500 ng/ml to about 1000 ng/ml, about 1000 ng/ml to about 1500 ng/ml, about 500 ng/ml to about 1500 ng/ml, about 1000 ng/ml to about 2000 ng/ml, about 1500 ng/ml to about 2000 ng/ml, about 2000 ng/ml to about 3000 ng/ml, about 2000 ng/ml to about 2500 ng/ml, about 2500 ng/ml to about 3000 ng/ml, about 3000 ng/ml to about 4000 ng/ml, about 3000 ng/ml to about 3500 ng/ml, about 3500 ng/ml to about 4000 ng/ml, about 4000 ng/ml to about 5000 ng/ml, about 4000 ng/ml to about 4500 ng/ml, about 4500 ng/ml to about 5000 ng/ml, about 5000 ng/ml to about 6000 ng/ml, about 5000 ng/ml to about 5500 ng/ml, about 5500 ng/ml to about 6000 ng/ml, about 6000 ng/ml to about 7000 ng/ml, about 7000 ng/ml to about 8000 ng/ml, or about 8000 ng/ml to about 9000 ng/ml.

The skilled artisan will appreciate that certain factors may influence the dosage and timing required to effectively treat a subject, including but not limited to, the severity of the medical disease or condition, previous treatments, the general health and/or age of the subject, and other diseases present. Moreover, treatment of a subject with a therapeutically effective amount of the therapeutic compositions described herein can include a single treatment or a series of treatments.

In some embodiments C16:1n7-palmitoleate, or derivatives or pharmaceutically acceptable salts thereof, are formulated as a pharmaceutical composition within a soft gelatin capsule. In some embodiments, the soft gelatin capsule includes about 0.5 grains, about 1 gram, about 1.5 grams, or about 2 grams of the pharmaceutical composition comprising at least 20%, at least 30%, at least 40%, at least 50%, at least 60%, at least 70%, at least 80% or at least 90% of C16:1n7-palmitoleate, or a derivative or pharmaceutically acceptable salt thereof. In some embodiments, one capsule per day is administered to a subject for the treatment or prevention obesity, the promotion of weight loss, and the support of weight management, as described herein. In some embodiments, two capsules per day are administered to the subject. In some embodiments, two to ten capsules per day are administered to the subject.

In some embodiments, the subject is a mammal, a reptile, or an amphibian. In some embodiments, the mammal is any mammal, including, for example, farm animals, such as sheep, pigs, cows, and horses; pet animals, such as dogs and cats; laboratory animals, such as rats, mice and rabbits. In some embodiments, the mammal is a human.

Combination Therapy with C16:1n7-Palmitoleate and Other Therapeutic Agents

In some embodiments, C16:1n7-palmitoleate, derivatives thereof pharmaceutically acceptable salts thereof, or combinations thereof, may be combined with one or more additional therapeutic agents for the prevention or treatment of obesity, the promotion of weight loss, and the support of weight management. By way of example, but not by way of limitation, treatment for obesity and promotion of weight loss, can include, in addition to C16:1n7-palmitoleate, derivatives thereof, pharmaceutically acceptable salts thereof, or combinations thereof, orlistat, lorcaserin, phentermine-topiramate, phendimetrazine, bupropion/naltrexone, methamphetamines, benzphetamine, cimetidine, human chorionic gonadotropin, mazindol, sibutramine, liraglutide, diethylpropion, topiramate, and phentermine.

In some embodiments, an additional therapeutic agent is administered to a subject in combination with C16:1n7-palmitoleate, its derivatives, pharmaceutically acceptable salts thereof, or combinations thereof, such that a synergistic therapeutic effect is produced. Therefore, lower doses of one or both of the therapeutic agents may be used in treating obesity and promoting weight loss, resulting in increased therapeutic efficacy and decreased side-effects.

In any case, the multiple therapeutic agents may be administered in any order, e.g., sequentially or separately or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary between more than zero weeks to less than four weeks. In addition, the combination methods, compositions and formulations are not to be limited to the use of only two agents.

### Kit of Parts

In one aspect, the present disclosure provides a kit of parts for the treatment of obesity and promotion of weight loss comprising a composition comprising C16:1n7-palmitoleate, derivatives thereof, pharmaceutically acceptable salts thereof, or a combination thereof and a means for administering the composition to a patient in need thereof. The means for administration can include, for example, C16:1n7-palmitoleate, a C16:1n7-palmitoleate, derivative, a C16:1n7-palmitoleate pharmaceutically acceptable salt, or any combination thereof, and a transdermal patch, a syringe, a needle, an IV bag comprising the composition, a vial comprising the composition, etc.

### EXAMPLES

The present technology is further illustrated by the following examples, which should not be construed as limiting in any way. For each of the examples below. C16:1n7-palmitoleate or any derivatives thereof could be used.

### Example 1: The Effect of Palmitoleic Acid (C16:1n7) on Lipogenesis

FIG. 1 illustrates de novo lipogenesis and β-oxidation in an adipocyte. As described in the examples below, in vitro experiments involving the administration of exogenous palmitoleic acid to bovine adipocytes downregulates de novo lipogenesis and upregulates fatty acid oxidation, thereby directing fatty acids towards energy expenditure and away from storage. Additionally, in vivo experiments involving the administration of exogenous palmitoleic acid to meat producing animals results in end-product inhibition that reduces de novo lipogenesis and decreases excess fat deposition.

Bovine adipocyte cultures were used to determine if addition of exogenous palmitoleic acid (C16:1n7) altered lipogenesis in vitro (Burns et al., 2012a). Adipocytes were harvested from the bovine cultures on day 6 and day 12 and analyzed using GLC and qPCR. Palmitoleic acid, cis-11 vaccenic acid (C18:1c11), cis-13 eicosenoic acid (C20:1c13), and total fatty acid levels showed a linear increase (P<0.05) in response to increasing concentrations of palmitoleic acid (50, 150 and 300 μM). In contrast, increasing concentrations of palmitoleic acid led to a decrease in palmitic acid (C16:0), stearic acid (C18:0), and oleic acid (C18:1) levels (P<0.05). FIG. 2 illustrates de novo lipogenesis and β-oxidation in an adipocyte after exogenous palmitoleic acid addition.

Palmitoleic acid treated cells showed a reduction in SCD-1, ELOVL6, and FASN mRNA expression levels compared to untreated control cells (P<0.05). The desaturation ratio of C18:1 cis-9/C18:0 decreased (P<0.05) in response to increasing concentrations of palmitoleic acid, which was consistent with the observed decrease in SCD-1 mRNA expression. Furthermore, experiments involving the administration of 2.5 mM [1-¹³C]acetate on day 6 demonstrated that palmitoleic acid supplemented cells had a lower rate of lipogenesis compared to controls.

These results demonstrate that addition of exogenous palmitoleic acid to adipocytes results in (a) an increase in the levels of palmitoleic acid and its elongation products, and (b) a reduction in SCD-1, FASN, and ELOVL6 levels. As such, C16:1n7-palmitoleate, derivatives thereof, pharmaceutically acceptable salts thereof, or a combination thereof, are useful in methods for decreasing de novo lipogenesis.

### Example 2: The Effect of Palmitoleic Acid (C16:1n7) on Fatty Acid Oxidation

The effect of palmitoleic acid supplementation on fatty acid oxidation in bovine adipocytes (see Kadegowda et al., 2011a) was examined by measuring the expression levels of Acyl-Coenzyme A oxidase 2 (ACOX2), Acyl-CoA dehydrogenase, long chain (ACADL), Phytanoyl-CoA 2-hydroxylase (PHYH), Caveolini (CAV1) and Adipose differentiation-related protein (ADEP). The geometric mean of Eukaryotic translation initiation factor 3, subunit k (EIF3K) and Ubiquitously expressed transcript (UXT) was used for normalization. Palmitoleic acid supplementation led to a 2.36-fold increase in ACADL (P≦0.07), and a 95.5-fold increase in PHYH (P<001), thus suggesting a potential increase in mitochondrial β-oxidation and peroxisomal α-oxidation, respectively.

These results demonstrate that palmitoleic acid regulates lipid metabolism in adipose tissue via mechanisms involving enhanced fatty acid oxidation. As such, C16:1n7-palmitoleate, derivatives thereof, pharmaceutically acceptable salts thereof, or a combination thereof, are useful in methods for increasing fatty acid oxidation.

### Example 3: The Effect of Palmitoleic Acid (C16:1177), Relative to Other Fatty Acids, on the Inhibition of Desaturation

In vitro studies utilizing ¹³C16:1 have confirmed that cis-11 vaccenic acid (C18:1cis11) and cis-13 eicosenoic acid (C20:1) are elongation products of palmitoleic acid (C16:1n7) in bovine adipocytes (Burns et al., 2012b). The experiments herein examined whether palmitoleic acid or its elongation products, cis-11 vaccenic acid (C18:1) and cis-13 eicosenoic acid (C20:1), are the active fatty acids that initiate the changes in lipogenesis that were observed in vitro. In addition, cells were incubated with ¹³C2 and ¹³C18:0 on day 6 to estimate lipogenic and desaturation rates using GLC-MS. In palmitoleic acid supplemented cells, C16: C18:1 cis-11, and C20:1 cis-13 levels were elevated compared with control cells (P<0.05). In C18:1 cis-11 supplemented cells, C18:1 cis-11 and C20:1 cis-13 levels were elevated compared to control cells (P<0.05). The C18:1 cis-9/C18:0 ratio was lower in palmitoleic acid supplemented cells compared with all other treatments (P<0.05). After 12 h of ¹³C18:0 incubation, cells supplemented with C16:1 had lower ¹³C18:1 cis-9 levels compared with all other treatments (P<0.05). Therefore, inhibition of desaturation and lipogenesis can be attributed to palmitoleic acid and not its elongation products.

Taken together, these results demonstrate that addition of exogenous palmitoleic acid downregulates enzymes involved in de novo lipogenesis (SCD-1, FASN, and ELOVL-6) and upregulates enzymes involved in β-oxidation (CTP-1 and ACADL). As such, C16:1n7-palmitoleate, derivatives thereof, pharmaceutically acceptable salts thereof, or a combination thereof, are useful in methods for reducing adipocyte hypertrophy and excess adipose tissue deposition.

### Example 4: Palmitoleic Acid (C16:1n7) Feeding Studies With Lambs

Randomized experiments were conducted to test the effect of feeding supplemental palmitoleic acid (C16:1n7) to obese lambs on lipogenesis and adipose tissue accretion. Fifteen Southdown wether lambs were used for this experiment. Lambs were fed traditional finishing diets designed to meet or exceed NRC (2007) requirements. When the lambs were estimated to have greater than 8 mm back fat as determined by real-time ultrasound (obese lamb model), three lambs were randomly selected for slaughter and their tissues collected (as described below) to serve as a pre-study control. The remaining lambs (n=12) were randomly assigned to one of two dietary treatments: 0 (placebo) or 7.5 mg/kg body weight of supplemental C16:1n7 daily for 28 d. For a 150 lb (68.2 kg) lamb, this level of supplementation will equate to 511.5 mg/hd/d or approximately 3 Cardia7 softgel capsules containing 210 mg C16:1n7 for a total of 630 mg/hd/d.

This level of palmitoleic acid supplementation was selected based on previous experiments where lambs were infused with varying levels (0, 2, or 5 mg/kg) of ¹³C16:1n7 to assess uptake and utilization in adipose tissues. In these experiments, the 5 mg/kg C16:1n7 level resulted in the greatest concentrations of ¹³C16:1n7 in serum and also altered blood glucose levels compared to control. This 5 mg/kg level is based on intravenous infusion levels and thus is adjusted for estimated ruminal bypass and intestinal absorption rates for dietary monounsaturated fatty acids for this oral feeding study.

At weekly intervals, blood samples were collected at pre- (0800) and post-feeding (1200) for determination of triglycerides, cholesterol, fatty acid composition, glycerol, glucose, non-esterified fatty acids, and insulin as described below. Real-time ultrasound measurements were taken weekly to estimate the amount of back fat thickness. At the end of the feeding period (28 d), lambs were slaughtered. Immediately after exsanguination and pelt removal, samples of subcutaneous adipose tissue at the 12th rib from the right side, breast, tail dock, mesenteric adipose tissue, pancreas and liver were flash frozen in liquid nitrogen and stored at −80° C. for subsequent RNA and protein extraction. Additional samples of subcutaneous fat at the 12th rib from the right side, breast, tail dock, mesenteric adipose tissue, and liver were also collected and stored at −20° C. for subsequent total lipid content, fatty acid composition, melting point, and cellularity analyses. The left side of each lamb carcass was divided into 3 sections (leg, middle, and shoulder). Each section was deboned and all soft tissues were ground. Weights were obtained on all tissues and bone. Ground tissues from each section were sampled for total lipid, crude protein, moisture and fatty acid composition in order to calculate a total body composition estimate (lean, fat, bone).

Blood lipids: Blood samples (6 ml in both EDTA and serum separator tubes) were taken weekly at pre- and post-feeding during the experimental feeding period (0, 7, 14, 21 and 28 d). Blood samples in EDTA tubes were collected on ice, immediately centrifuged, and frozen for further analysis. Glycerol, glucose, cholesterol, HDL-cholesterol, and triglyceride levels in plasma were analyzed by an enzymatic method (BioVision, San Francisco, Calif.). Plasma nonesterified fatty acids (NEFA) were measured using a colorimetric assay (Wako, Richmond, Va.) and insulin was measured using an ovine ELISA kit (Mercodia, Winston Salem, N.C.). These measurements were used to assess lipid oxidation according to Pittard et al., (2010). Blood glucose levels were measured immediately at the time of collection using AlphaTrak Glucose Meter. Blood samples were allowed to clot overnight and centrifuged at 1,000×g for 20 min at 4° C. for serum collection. Serum samples were lyophilized and transmethylated using the method described in Park and Goins (1984). Fatty acid methyl esters were separated on a gas chromatograph using a Supelco SP-2560 capillary column with a split ratio of 5:1. The addition of internal standard (C23:0) allows for quantification of fatty acid weight percentages as well as gravimetric amounts (Duckett et al., 2002; Miller et al., 2009).

Tissue lipids: Adipose cellularity was determined in duplicate samples by osmium tetroxide fixation according to Mersmann and MacNeil (1986) and Pavan and Duckett (2007). The remaining adipose tissue samples were lyophilized, ground in a food processor, and stored at −80° C. Proximate analyses (moisture, protein, ash and mineral content) was conducted at the Clemson University Agricultural Services Laboratory. Total fat content of the ground samples was determined using Ankom XT-15 total fat extractor in triplicate. Lipids were transmethylated according to Park and Goins (1984) and analyzed by GLC (Duckett et al., 2002). Melting point of the fat samples were determined using the OptiMelt Automated Melting Point System (Stanford Research Systems, Sunnyvale, Calif.) according to Duckett et al., (2010).

qPCR: RNA was extracted from adipose and liver tissues according to the method of Duckett et al., (2009). RNA quantity and quality were determined using the Nanodrop 1000 and Agilent Bioanalyzer 2100. Real-time gene expression assays were conducted according to Duckett et al., (2009) using ovine primers for SCD-1, FASN, ACC, ELOVL6, ACADL, and CPT-1a & b (Price et al., 2003).

Immunoblot analysis: Microsomes and cytosol fractions from subcutaneous adipose tissue and liver samples were isolated by differential centrifugation according to Schenkman and Ciniti (1978) as modified by Doran et al., (2006). Abundance of microsomal protein (SCD-1) and cytosolic proteins (ACC and FASN) were estimated by western blotting (Pratt et al., 2010).

Statistical Analysis: Analysis of variance was generated using a mixed model procedure with dietary palmitoleic acid level (0 or 5 mg/kg BW) as the fixed effect, with lamb as the experimental unit. Plasma and serum measurements were analyzed using repeated measures analysis. Least squares means were computed and separated statistically using Fisher's Protected LSD test. Relative gene expression was analyzed using Pair-wise Fixed Reallocation Randomization Test (Pfaffl et al., 2002).

Results: Southdown wethers (n=15; 95 kg BW) were used to assess the effects of palmitoleic acid (C16:1n7) infusion on body composition and adipocyte cell size in obese sheep. Omega-7 enriched oil (45% palmitoleic acid) was infused, twice daily for 28 days via Indwelling jugular catheter at three concentrations: 0 (CON), 5 (MED) or 10 (HI) mg/kg BW⁻¹/d⁻¹. The oil was solubilized in 40% ethanol and immediately injected into the catheter at 0800 and 1600 h for each lamb. All lambs received the same amount of 40% ethanol per dose regardless of oil level. Blood samples were collected at 5 minutes post dosing on a weekly basis to assess uptake of palmitoleic acid into circulation. After 28 days, lambs were slaughtered. At slaughter, weights of omental and mesenteric adipose tissue were collected as well as hot carcass weight. At 24 hr postmortem, carcass data was collected and samples were obtained from subcutaneous and intramuscular adipose tissues for cell size determination. Serum palmitoleic acid levels (mg/mL) at 5 minutes post injection were 60% higher in HI compared to CON (P<0.05). Serum palmitoleic acid levels also tended to be higher 31%) in MED compared to CON (P=0.09). Serum cis-11 vaccenic acid levels were also elevated in HI compared to CON (P<0.05), which increased over time for HI but not in CON (P<0.05). The ratio of C16:1 to C16:0 was elevated in both the MED and HI treatments (P<0.05). Average daily weight gain during the 28 day treatment period was lowered by 76% for HI compared to CON (P<0.05). Carcass parameters and visceral adipose depots were not different between treatments (P>0.05). Mean subcutaneous adipocyte size did not differ (P>0.05) between treatments and averaged 92.2 μm. Mean intramuscular adipocyte size was reduced (P<0.05; 66.1 vs. 74.2 μm) in HI compared to CON.

These results demonstrate that administration of an omega-7 enriched oil to obese sheep increased circulating C16:1n7 levels, reduced average daily weight gains, and decreased intramuscular adipocyte size. As such, C16:1n7-palmitoleate, derivatives thereof, pharmaceutically acceptable salts thereof, or a combination thereof, are useful in methods of treating or preventing obesity, methods of promoting weight loss, and methods of supporting weight management.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to he understood that the terminology used herein is for the purpose of describing particular embodiments only, and is not intended to he limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

### REFERENCES

Barber, M. C., R. J. Ward, S. E. Richards, A. M. Salter, P. J. Buttery, R. G. Vernon, and M. Travers. 2000. Ovine adipose tissue monounsaturated fat content is correlated to depot-specific expression of the stearoyl-CoA desaturase gene. J. Anim. Sci. 78:62-68.

Bernard, L., C. Leroux, H. Hayes, M. Gautier, Y. Chilliard, and P. Martin, 2001. Characterization of the caprine stearoyl-CoA desaturase gene and its mRNA showing an unusually long 3′-UTR sequence arising from a single exon. Gene 281:53-61.

Bernlohr, D. A., M. A. Bolanowski, T. J. Kelly, Jr., and M. D. Lane. 1985. Evidence for an increase in transcript of specific mRNAs during differentiation of 3T3-L1 preadipocytes. J. Biol. Chem. 260:5563-5567.

Burns, A. P., S. K. Duckett, and S. L. Pratt. 2012a. Palmitoleic acid (C16:1) changes fatty acid profiles and alters gene expression in bovine adipocytes cultures.

Burns, T. A., S. K. Duckett, S. L. Pratt, and T. C. Jenkins. 2012b. Palmitoleic acid (C16:1), not an elongation product, decreases lipogenesis and desaturation in bovine adipocyte cultures. Lipids (In press).

Canovas, A., J. Estany, M. Tor, R. N. Pena, and O. Doran. 2009. Acetyl-CoA carboxylase and stearoyl-CoA desaturase protein expression in subcutaneous adipose tissue is reduced in pigs selected for decreased backfat thickness at constant intramuscular fat content. J. Anim. Sci. 87:3905-3914.

Cao, H., K. Gerhold, J. R. Mayers, M. M. Wiest, S. M. Watkins, and G. S. Hotamisligil. 2008. Identification of a lipokine, a lipid hormone linking adipose tissue to systemic metabolism. Cell. 134:933-944.

Casmir, D. A. and J. M. Ntambi, 1996. cAMP activated the expression of stearoyl-CoA desaturase gene I during early preadipocyte differentiation. J. Biol. Chem 271:29847-29853.

Chung, M., S. Ha, S. Jeong, J. Bok, K. Cho, M. Baik, and Y. Choi. 2000. Cloning and characterization of bovine stearoyl-CoA desaturase 1 cDNA. from adipose tissues. Biosci. Biotechnol. Biochem. 64:1526-1530.

Doran, O., S. K. Moule, G. A. Teye, F. M. Whittington, K. G. Hallett, and J. D. Wood. 2006. A reduced protein diet induces stearoyl-CoA desaturase protein expression in pig muscle but not in subcutaneous adipose tissue: relationship with intramuscular lipid formation. Brit. J. Nutr. 95:609-617.

Duckett, S. K., E. Pavan, and S. L. Pratt. 2009. Com oil or corn grain supplementation to steers grazing endophyte-free tall fescue. II. Effects on subcutaneous fatty acid content and. lipogenic gene expression. J. Anim. Sci, 87:1120-1128.

Duckett, S. K., J. G. Andrae, and F. N. Owens. 2002. Effect of high-oil corn or added corn oil on ruminal biohydrogenation of fatty acids and conjugated linoleic acid formation in beef steers fed finishing diets. J. Anim. Sci. 80:3:353-3360.

Duckett, S. K., J. P. S. Neel, W. Swecker, J. P. Fontenot, and W. Clapham. 2010. Effect of finishing system on subcutaneous fat melting point and fatty acid composition. J. Anim. Sci. 88 (E-Suppl. 2):68 (Abstr.)

Enoch, H. G., A. Catala, and P. Strittmatter. 1976. Mechanism of rat microsomal stearoyl-CoA desaturase. J. Biol. Chem. 251:5095-5103.

Flowers, M. I. and J. M Natinbi. 2009. Stearoyl-CoA desaturase and its relation to high-carbohydrate diets and obesity. Biochim. Biophys. Acta 1791:85-91.

Folch, J., M. Lees, and G. H. Sloan-Stanley. 1957. A simple method for the isolation and purification of total lipides from animal tissues. J. Biol. Chem. 226:497-509

Hirai, S., H. Matsumoto, N. Hino, H. Kawachi, T. Matsui, and H. Yano. 2007. Myostatin inhibits differentiation of bovine preadipocytes. Dom. Anim. Endocr. 32:1-14.

Hood, R. L. and C. E. Allen. 1973. Cellularity of bovine adipose tissue. J. Lipid Res. 14:605-610.

Ingle, D. L., D. E. Bauman, and U. S. Garrigus. 1972a. Lipogenesis in the ruminant: in vitro study of tissue sites, carbon source, and reducing equivalent generation for fatty acid synthesis. J. Nutr. 102:609-616.

Ingle, D. L., D. E. Bauman, and U. Garrigus. 1972b. Lipogenesis in the ruminant: in vivo site of fatty acid synthesis in sheep. J. Nutr. 102:617-624.

Ichihara, K., K. Yoneda, A. Takahashi, N. Hoshino, and M. Matsuda. 2011. Improved methods for the fatty acid analysis of blood lipid classes. Lipids 46:297-306.

Kadegowda, A., S. K. Duckett, T. A. Burns, and S. L. Pratt. 2011a. Palmitoleic acid regulation of lipid metabolism in primary bovine adipocytes could involve genes related to fatty acid oxidation. J. Anim. Sci. (Abstr.; In press).

Kadegowda, A., S. K. Duckett, T. A. Burns, and S. L. Pratt. 2011b. Effect of stearoyl-CoA desaturase-1 (SCD-1) inhibitors on lipid metabolism and cellular proliferation in primary bovine adipocytes. J. Anim. Sci. (Abstr.; In press).

Klingenberg, I. L., D. A. Knabe, and S. B. Smith. 1995. Lipid metabolism in pigs fed beef tallow or high oleic acid sunflower oil. Comp. Biochem. Physiol. 110B:183-192.

Lengi, A. J. and B. A. Corl. 2008. Comparison of pig, sheep, and chicken SCDS homologs: evidence for an early gene duplication event. Comp. Biochem. Physiol. B Biochem. Mol. Biol. 150:440-6.

Lengi, A. J. and B. A. Corl. 2010. Factors influencing the differentiation bovine preadipocytes in vitro. J. Anim. Sci. 88:1999-2008.

Martin, G. S., D. K. Lunt, K. G. Britain, and S. B. Smith. 1999. Postnatal development of stearoyl coenzyme A desaturase gene expression and adiposity in bovine subcutaneous adipose tissues, J. Anim. Sci. 77:630-636.

Mersmann, H. J. and M. D. MacNeil. 1986. Variables in estimation of adipocyte size and number with a particle counter. J. Anim. Sci. 62:980-991.

Miller, M., J. Andrae, S. Duckett. and S. Pratt 2009. Management strategies to improve development of replacement heifers on tall fescue-based systems. J. Anim. Sci. 87 (E-Suppl. 1):17 (Abstr.).

Ntambi, J. M. 1992. Dietary regulation of stearoyl-CoA desaturase-1 gene expression in mouse liver. J. Biol. Chem. 267:10925-10930.

Ntambi, J. M., S. A. Buhrow, K. H. Kaestner, R. J. Christy, E. Sibley, T. J. Kelly, and M. D. Lane. 1988. Differentiation-induced gene expression in 3T3-L1 preadipocytes. Characterization of a differentially expressed gene encoding stearoyl-CoA desaturase. J. Biol. Chem. 25:17291-17300.

Ntambi, J. M., M. Miyazaki, J. P. Store, H. Lan, C. M. Kendziorski, B. S. Yandell, Y. Song, P. Cohen, and J. M. Friedman, and A. D. Attie. 2002. Loss of stearoyl-CoA desaturase-1 function protects mice against adiposity. Proc. Natl. Acad. Sci. 99:11482-11486.

Park, P. and R. E. Goins. 1984. In situ preparation of fatty acid methyl esters for analysis of fatty acid composition in food. J. Food Sci. 59:1262-1266.

Pavan, E. and S. K. Duckett. 2007. Corn oil supplementation to steers grazing endophyte-free tall fescue. II. Effects on longissimus muscle and subcutaneous adipose fatty acid composition and stearoyl-CoA desaturase activity and expression. J. Anim. Sci. 85:1731-1740.

Pfaffl, M. W., G. W. Horgan and L. Dempfle. 2002. Relative expression software tool (REST) for group-wise comparison and statistical analysis of relative expression results in Real-Time PCR. Nucl. Acids. Res. 30:E36.

Pittard, F., V. Van Wymelbeke, E. Garrigue, C. Moro, F. Crampes, J. C. Guilland, M. Berlan, I. De Glisezinski, I. Harant, D. Riviere, and L. Brondel. 2010. Lipid oxidation in overweight men after exercise and food intake. Metab. Clin. Expt. 59:267-274.

Pratt, S. L., C. K. Ogle, J. X. Mao, W. Zhao, G. Lovell and N. D. Horseman. 2000. Interleukin-6 signal transduction in human intestinal epithelial cells. Shock. 13:435-440.

Pratt, S. L., T. A. Burns, and S. K. Duckett. 2010. Expression of MicroRNA During Early Differentiation of Bovine Adipocytes. J. Nucleic Acid Invest. 1:e12.

Price, N. T., V. N. Jackson, F. R. van der Leij, J. M Cameron, M. T. Travers, B. Bartelds, N. C. Huikman, and V. A. Zammit. 2003. Cloning and expression of the liver and muscle isoforms of ovine carnitine palmitoyltransferase 1: residues within the N-terminus of the muscle isoform influence the kinetic properties of the enzyme. Bioch. J. 372:871-879.

Reeves, L. M., M. L. Williams, and T. C. Jenkins. 1998. In vitro biohydrogenation and total tract digestibility of oleamide by sheep, J. Sci. Food Agric. 77:187-192.

Ren, J., C. Knorr, L. Huang, and B. Brenig. 2004. Isolation and molecular characterization of the porcine stearoyl-CoA desaturase gene. Gene 340:19-30.

Robelin, J. 1986. Growth of adipose tissues in cattle: partitioning between depots, chemical composition and cellularity. A review. Livest. Prod. Sci. 14:349-364. Schenkman, J. B. and D. L. Cinti. 1978. Preparation of microsomes with calcium.

Methods Enzymol 52:83-89.

Smith, S. B., H. J. Mersmann, E. O. Smith, and K. G. Britain. 1999. Stearoyl-CoA desaturase gene expression during growth in adipose tissues from obese and crossbred pigs. J. Anim. Sci. 77:1710-1716.

St. John, L. C., D. K. Lunt, and S. B. Smith. 1991. Fatty acid elongation and desaturation enzyme activities of bovine liver and subcutaneous adipose tissue microsomes. J. Anim. Sci. 69:1064-1073.

Ward, R. J., M. T. Travers, S. E. Richards, R. G. Vernon, A. M. Salter, P. J. Buttery, and M. C. Barber. 1998. Stearoyl-CoA desaturase mRNA is transcribed from a single gene in the ovine genome. Biochim. Biophys. Acta 1391:145-156.

Zhang, L., L. Ge, S. Parimoo, K. Stenn, and S. M. Prouty. 1999. Human stearoyl-CoAdesaturase: alternative transcripts generated from a single gene by usage of tandem polyadenylation sites. Biochem. J. 340:255-264.

What is claimed is:

1.-18. (canceled)

19. A method for promoting weight loss and supporting weight management in a subject in need thereof, the method comprising administering to the subject an effective amount of a composition, wherein the composition comprises a C16:1n7-palmitoleate derivative.

20. The method of claim 19, wherein the composition comprises at least about 10 wt % of the C16:1n7-palmitoleate derivative.

21. The method of claim 19, wherein the composition comprises at least about 20 wt % of the C16:1n7-palmitoleate derivative.

22. The method of claim 19, wherein the composition comprises at least about 50 wt % of the C16:1n7-palmitoleate derivative.

23. The method of claim 19, wherein the composition comprises at least about 90 wt % of the C16:1n7-palmitoleate derivative.

24. The method of claim 19, wherein the C16:1n7-palmitoleate derivative is a (C₁-C₈)alkyl ester.

25. The method of claim 19, wherein the C16:1n7-palmitoleate derivative is an ethyl ester.

26. The method of claim 19, wherein the C16:1n7-palmitoleate derivative is a methyl ester.

27. The method of claim 19, wherein the C16:1n7-palmitoleate derivative is a free acid or a pharmaceutically acceptable salt thereof.

28. The method of claim 19, wherein the C16:1n7-palmitoleate derivative is selected from the group consisting of monoglycerides, diglycerides, triglycerides and combinations thereof.

29. The method of claim 19, wherein the C16:1n7-palmitoleate derivative is a cis-C16:1n7-palmitoleate derivative.

30. The method of claim 19, wherein the C16:1n7-palmitoleate derivative comprises a C16:1n7-palmitoleate moiety that is obtained from fish.

31. The method of claim 30, wherein the fish is selected from the group consisting of anchovies, menhaden, pollock, herring, cod, salmon, smelt, tuna, mackerel, krill and a combination thereof.

32. The method of claim 30, wherein the fish is anchovies.

33. The method of claim 30, wherein the fish is menhaden.

34. The method of claim 19, wherein the subject is obese or overweight with a body mass index over 25.

35. The method of claim 34, wherein the body mass index of the subject is reduced to 18.5-25 following the administration of the composition to the subject.

36. The method of claim 35, wherein the body mass index of the subject is maintained within the range of 18.5-25 with further administrations of the composition to the subject.
